(19) United States
(12) Patent Application Publication
(10) Pub. No.: US 2026/0020973 A1
(43) Pub. Date: Jan. 22, 2026

(54) MANDIBULAR ADVANCEMENT DEVICE WITH A SUPPORT BAFFLE

(71) Applicant: DCSTAR INC, New York, NY (US)

(72) Inventor: David LUO, New York, NY (US)

(21) Appl. No.: 18/776,963

(22) Filed: Jul. 18, 2024

(51) Int. Cl. A61F 5/56

(52) U.S. Cl. CPC A61F 5/566

(57) ABSTRACT

A mandibular advancement device, specifically a device configured to enhance longevity, therapeutic effectiveness, and user comfort. The mandibular advancement device consists of an upper tray assembly and a lower tray assembly. The upper tray assembly includes an upper moldable component and an upper frame, while the lower tray assembly comprises a lower moldable component and a lower frame. The mandibular advancement device includes a support baffle on the lower frame, which is configured to be positioned near the tongue side of the lower frame during use. Various parameters of the support baffle are defined to achieve a mandibular advancement device that is more suitable for patients compared to existing devices on the market.

### TECHNICAL FIELD

The present disclosure relates to a mandibular advancement device with a support baffle, particularly configured to prevent, reduce, or eliminate snoring and/or obstructive sleep apnea.

### BACKGROUND

Sleep Apnea Syndrome (SAS) is one of the most common sleep and breathing disorders, with Obstructive Sleep Apnea/Hypopnea Syndrome being the most prevalent type. Obstructive sleep apnea occurs when the airway is fully or partially blocked by relaxed muscles and other tissues during sleep. This blockage can be caused by various factors, including but not limited to soft tissue relaxation, pharyngeal muscle relaxation, uvula and tongue abnormalities. Currently, non-surgical treatments are more widely accepted by patients, and using a mandibular advancement device is one of the non-surgical treatment methods.

A mandibular advancement device is similar to a mouthguard with tooth protection functionality. Patients wear the mandibular advancement device on their teeth while sleeping. The fundamental principle is to use the anterior-posterior displacement fixation of the upper and lower parts of the device to move the patient's mandible (i.e., lower jaw) in the opposite direction of the tongue, creating more space at the back of the throat. This prevents the airway from being blocked by the backward displacement of the tongue and the collapse of the surrounding soft tissues during sleep. Moreover, the mandibular advancement device only needs to be placed in the patient's mouth while sleeping, without causing irritation to the skin or hair, and without restricting the patient's movement. It is simple, compact, and easy to carry. For patients already accustomed to wearing a mouthguard-type mandibular advancement device, this device is a more readily accepted treatment method compared to other options.

Although the mandibular advancement device has many advantages as mentioned above, in order to perfectly match the patient's teeth and jaw, the manufacturing process requires a customized model based on the patient's teeth or oral cavity. Customized mandibular advancement devices use advanced manufacturing equipment and a complete digital manufacturing process, which can result in fewer side effects for the patient but are expensive and time-consuming. Therefore, customized mandibular advancement devices are not the optimal choice for the general public. Additionally, some customized mandibular advancement devices still require the involvement of professionals, which increases the time cost for patients.

### SUMMARY

In view of the above shortcomings, the present disclosure provides a mandibular advancement device that is convenient for patients to use, reduces the cost for patients, and is comfortable to wear.

In one embodiment, a mandibular advancement device with a support baffle is provided. The mandibular advancement device with a support baffle includes an upper tray assembly and a lower tray assembly. The upper tray assembly is configured to have a generally arcuate outer shape to conform to an upper dental arch curve of a patient, and the lower tray assembly is configured to have a generally arcuate outer shape to conform to a lower dental arch curve of the patient. The upper tray assembly includes an upper moldable component and an upper frame. The lower tray assembly includes a lower moldable component and a lower frame. The lower frame includes a support baffle, configured to be positioned on the lower frame near a tongue side during use. The support baffle has a top edge and a bottom edge, and a line connecting intersections of the top edge and the bottom edge with a same sagittal plane of the lower frame forms an angle with a horizontal plane between 20° to 150°.

In one embodiment, the arcuate shapes of the upper moldable component and the lower moldable component include at least one of an ovoid arch shape, a conical arch shape, and a square arch shape.

In one embodiment, the upper moldable component and the lower moldable component are configured to at least partially conform to the upper dental arch and the lower dental arch of the patient during use, respectively.

In one embodiment, the length of the bottom edge of the support baffle is greater than 10% of an overall curve length at a bottom edge of the lower frame.

In one embodiment, the upper moldable component and the lower moldable component include a flexible thermoplastic material.

In one embodiment, the hardness of a material of the upper frame and the lower frame is at least partially greater than a hardness of the material of the upper moldable component and the lower moldable component.

In another embodiment, a mandibular advancement device with a support baffle is provided. The mandibular advancement device with a support baffle includes an upper tray assembly and a lower tray assembly. The upper tray assembly is configured to be positioned adjacent to an upper dental arch of a patient when located in the patient's oral cavity. The lower tray assembly is configured to be positioned adjacent to a lower dental arch of the patient when located in the patient's oral cavity. The upper tray assembly includes an upper moldable component and an upper frame. The lower tray assembly includes a lower moldable component and a lower frame. The lower frame includes a support baffle, configured to be positioned on the lower frame near a tongue side during use. In an orthogonal view from below, the support baffle has a shaded area, and a ratio of the shaded area to an area of the lower frame in the orthogonal view from below is less than or equal to 2:5.

In one embodiment, the support baffle is divided into multiple spaced-apart sections.

In one embodiment, the lower moldable component has a shape that engages and matches the support baffle.

In one embodiment, the upper moldable component and the lower moldable component are configured to soften when heated, to allow them to be adjusted and molded to respectively match the upper dental arch and the lower dental arch of the patient upon biting down.

In one embodiment, the upper moldable component and the lower moldable component include a moldable material having a deformation temperature of less than 100 degrees Celsius.

In one embodiment, the upper frame and the lower frame include a high-temperature stable material having a deformation temperature greater than 100 degrees Celsius.

In one embodiment, a mandibular advancement device with a support baffle is provided. The mandibular advancement device with a support baffle includes an upper tray assembly and a lower tray assembly. The upper tray assembly is configured to be positioned adjacent to an upper dental arch of a patient when located in the patient's oral cavity. The lower tray assembly is configured to be positioned adjacent to a lower dental arch of the patient when located in the patient's oral cavity. The upper tray assembly includes an upper moldable component and an upper frame. The lower tray assembly includes a lower moldable component and a lower frame. The lower frame includes a support baffle, configured to be positioned on the lower frame near a tongue side during use. In an orthogonal view from below, the support baffle has a shaded area, and a ratio of the shaded area to an area of the lower frame in the orthogonal view from below is less than or equal to 2:5. The support baffle has a shape that is higher in the middle and lower on both sides.

In one embodiment, the upper frame and the lower frame are configured to engage and support the upper moldable component and the lower moldable component, respectively.

In one embodiment, a position adjustment module is provided between the upper frame and the lower frame.

In one embodiment, the position adjustment module has two or more adjustment positions.

In one embodiment, a maximum height of the support baffle is less than or equal to a maximum height of the lower moldable component.

In one embodiment, a mandibular advancement device with a support baffle is provided. The mandibular advancement device with a support baffle includes an upper tray assembly and a lower tray assembly. The upper tray assembly is configured to be positioned adjacent to an upper dental arch of a patient when located in the patient's oral cavity. The lower tray assembly is configured to be positioned adjacent to a lower dental arch of the patient when located in the patient's oral cavity. The upper tray assembly includes an upper moldable component and an upper frame. The lower tray assembly includes a lower moldable component and a lower frame. The lower frame includes a support baffle, configured to be positioned on the lower frame near a tongue side during use. The support baffle has one or more of the following characteristics: having a top edge and a bottom edge, and a line connecting intersections of the top edge and the bottom edge with a same sagittal plane of the lower frame forms an angle with a horizontal plane between 20° to 150°; a length of the top edge of the support baffle being greater than or equal to 5 mm; a thickness of the support baffle being between 0.3 mm to 10 mm; a vertical height of the support baffle being less than or equal to 15 mm; and a hardness of the support baffle being between Shore D45 to Shore D95.

In one embodiment, the top edge and the bottom edge of the support baffle have continuous curvature.

In one embodiment, the upper tray assembly and the lower tray assembly include at least two different materials.

In one embodiment, a projected area of the upper moldable component and the lower moldable component on the horizontal plane is greater than a projected area of the upper frame and the lower frame on the horizontal plane.

In one embodiment, the lower moldable component has a shape that engages and matches the support baffle.

The mandibular advancement device of the present disclosure has at least the following beneficial effects:

(1) Using a mandibular advancement device to prevent or reduce snoring and/or obstructive sleep apnea during sleep is a good conservative therapy alternative to CPAP. The current market offers various mandibular advancement devices with different designs, classified into moldable and non-moldable mandibular advancement devices based on their adjustability and adaptability during use. This disclosure pertains to a moldable mandibular advancement device. Non-moldable mandibular advancement devices require customization under the guidance of professional doctors. Since they are customized, they can perfectly match the patient's teeth and oral conditions, providing better comfort for the patient. However, due to the need for professional guidance and adjustment for the customized devices, the treatment cost of non-moldable mandibular advancement devices is relatively high, making them expensive. For some patients, the process of coordinating with doctors and customization is relatively complex and troublesome. Compared to non-moldable mandibular advancement devices, these patients, as well as those with limited budgets, might prefer purchasing moldable mandibular advancement devices. Moldable mandibular advancement devices allow patients to achieve a customized fit through a simple heating and biting process. They are relatively cheaper than customized devices, making them a more economical choice for most patients.

(2) Although moldable mandibular advancement devices are more likely to be chosen by most patients compared to non-moldable ones, they still have significant problems and limitations. Currently, patient reviews for moldable mandibular advancement devices on the market are generally poor, with low satisfaction levels mainly due to the need for improvements in comfort and reliability. For example, the primary function of the lower tray assembly is to pull the patient's mandible away from the tongue side. However, because this goes against the force of inertia, the patient's mandible exerts a force on the lower tray assembly in the opposite direction of the mandibular advancement device's traction force. Existing mandibular advancement devices without support baffles lack structures that provide support to the upper and lower moldable components to counteract the force exerted by the mandible on the lower moldable component. Since the upper and lower moldable components include flexible materials, this force easily causes deformation, particularly in the lower moldable component. Deformation of the lower moldable component leads to the backward sliding of the mandible, rendering the device ineffective. Additionally, some current mandibular advancement devices tend to dislodge at the connection points between the upper and lower frames and the upper and lower moldable components, especially in the lower tray assembly. The lower moldable component and the lower frame are often connected by small snap-fitting structures, resulting in an insecure attachment. Due to the force exerted by the mandible on the lower moldable component and the flexibility of its flexible material, these snap-fittings can easily detach, causing separation between the lower moldable component and the lower frame. This disclosure, through comparison with existing designs, innovatively introduces a mandibular advancement device with a support baffle, which prevents the separation of the lower moldable component from the lower frame. Compared to existing mandibular advancement devices on the market, this innovative design offers greater convenience for patients during use and facilitates better patient compliance. This key innovation addresses critical issues in the current technology, providing an advantageous and novel solution.

(3) For patients, the mandibular advancement device of this disclosure not only prevents the displacement of the lower moldable component from the lower frame but also includes an angled support baffle. Typically, the gums and lower teeth are not vertical but have a sloped profile. The angled support baffle matches the slope of the gums and teeth, allowing the device to fit more closely to the patient's oral cavity and providing greater comfort during use. Comfort is a crucial factor for patients when purchasing a device. If the device is uncomfortable or affects treatment by causing issues such as component detachment, patients may be reluctant to wear it for extended periods, thus impacting treatment efficacy. Therefore, this disclosure improves patient compliance to some extent, ensuring more stable treatment. Moreover, the disclosure specifies various parameters to enhance the effectiveness of the support baffle during use. For instance, by defining the angle and height of the support baffle, the device can be made suitable for a wide range of patients. The angle of the support baffle is set within a range of 20° to 150°, preferably 45° to 90°, and the height is set to be less than or equal to 15 mm. If the support baffle extends outward at too great an angle or is too tall, it may exceed its basic position and restrict the occlusion of the upper and lower jaws. Conversely, if the support baffle is angled inward too much or is too short, it may lose its effectiveness. This disclosure also specifies the length of the support baffle as needed. Since the actual growth pattern of teeth and gums is higher in the middle and lower on both sides, and due to the autonomous movement of the patient's mandible towards the tongue side, the force exerted on the lower tray assembly mainly concentrates in the middle part. Therefore, placing the support baffle in the middle section of the lower frame is the optimal arrangement. If the baffle extends too far on both sides, it essentially becomes an ineffective area. Additionally, setting the hardness of the support baffle is a crucial aspect. As long as the support baffle is safe for the patient, no specific material requirements are imposed, allowing the baffle to be made of any material that complies with safety regulations. However, to ensure the support baffle functions effectively as part of the mandibular advancement device, it must be able to support the lower moldable component. Given this requirement, the hardness of the support baffle is specified to be between Shore D45 to Shore D95. Within this range, overly hard support baffles can injure the patient's oral cavity, while overly soft baffles cannot provide adequate support. Therefore, the preferred hardness range is Shore D65 to Shore D85, making it a more suitable choice for use. All data in this disclosure are selected through multiple measurements with professional machines, providing scientifically based support baffle, ensuring both effectiveness and comfort for the patient. This effective design of the support baffle distinguishes it from existing mandibular advancement devices, offering superior performance and a better experience for patients.

(4) For the product itself, mandibular advancement devices without a support baffle often cause the lower moldable component to detach, and because the upper and lower moldable components include flexible materials, they are highly susceptible to damage. Damage to the product not only reduces the lifespan of the mandibular advancement device but also leads to instability, thereby diminishing its therapeutic effectiveness. The mandibular advancement device with a support baffle of this disclosure, significantly enhances the stability of the device by reinforcing the fixation between the lower moldable component and the lower frame. This design effectively prevents opposing forces from pulling on the lower moldable component, ensuring it does not easily detach or get damaged during use. This improvement not only makes the mandibular advancement device more durable but also ensures the stability and reliability of its therapeutic effect.

### BRIEF DESCRIPTION OF THE DRAWINGS

FIG. 1 is an overall schematic diagram of a mandibular advancement device according to one embodiment of the present disclosure;

FIGS. 2A, 2B, and 2C are top views showing the upper tray assembly and the shapes of the upper dental arch according to several embodiments of the present disclosure;

FIG. 3 is an exploded view schematic diagram showing the structure of the mandibular advancement device according to one embodiment of the present disclosure;

FIG. 4 is a top view of the upper moldable component and upper frame according to one embodiment of the present disclosure;

FIG. 5 is an overall schematic diagram of the lower frame according to one embodiment of the present disclosure;

FIG. 6 is a schematic diagram showing the sliding of the lower moldable component and the lower frame during use according to one embodiment of the present disclosure;

FIG. 7 is an exploded view schematic diagram of the lower moldable component and the lower frame according to one embodiment of the present disclosure;

FIG. 8 is an overall schematic diagram of a mandibular advancement device including the upper frame and the lower frame, each of which has a support baffle according to one embodiment of the present disclosure;

FIG. 9 is a schematic diagram of horizontal, sagittal, and coronal planes according to several embodiments of the present disclosure;

FIG. 10 is an overall schematic diagram and cross-sectional view of the lower frame intersecting with a same sagittal plane according to one embodiment of the present disclosure;

FIG. 11 is a schematic diagram of the lower tray assembly occluding with the mandible according to one embodiment of the present disclosure;

FIGS. 12A and 12B are schematic diagrams showing the lower tray assembly with support baffles at different angles during use according to one embodiment of the present disclosure;

FIG. 13 is an overall schematic diagram of the lower frame concerning the support baffle data according to the first embodiment of the present disclosure;

FIGS. 14A, 14B, and 14C are overall schematic diagrams of the lower frame with different types of support baffles according to one embodiment of the present disclosure;

FIG. 15 is a top view of the lower frame according to one embodiment of the present disclosure;

FIG. 16 is an overall schematic diagram of the lower frame with different types of support baffles according to the another embodiment of the present disclosure;

FIG. 17 is a schematic diagram showing the hardness testing of support baffles according to several embodiments of the present disclosure.

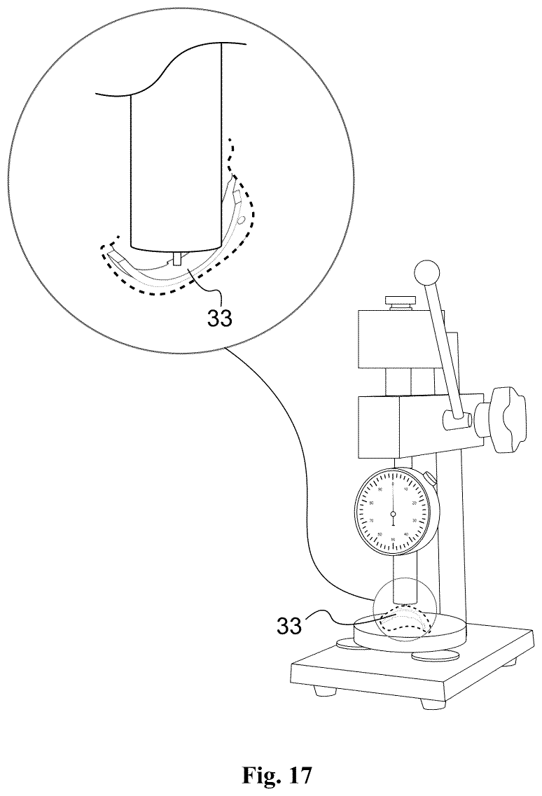

### DETAILED DESCRIPTION

To make the objectives, features, and advantages of the present disclosure more apparent and understandable, the specific embodiments of the disclosure are described in detail below in conjunction with the accompanying drawings. Many specific details are set forth in the following description to provide a thorough understanding of the disclosure. However, the disclosure can be implemented in many different ways other than those described here, and those skilled in the art can make similar modifications without departing from the spirit of the disclosure. Therefore, the disclosure is not limited to the specific embodiments disclosed below.

Compared to existing mandibular advancement devices on the market, the present disclosure innovatively introduces a structure of a support baffle 33. Through the design and use of the support baffle, the performance of the mandibular advancement device is maximized, thereby enhancing its effectiveness and the comfort of use for patients. This results in superior effectiveness, comfort, reliability, and durability. Thus, the mandibular advancement device 1 of the present disclosure not only improves performance metrics but also provides patients with a more stable, safe, and comfortable experience.

The present disclosure provides a mandibular advancement device that is convenient for patients to use, reduces their costs, and is comfortable to wear.

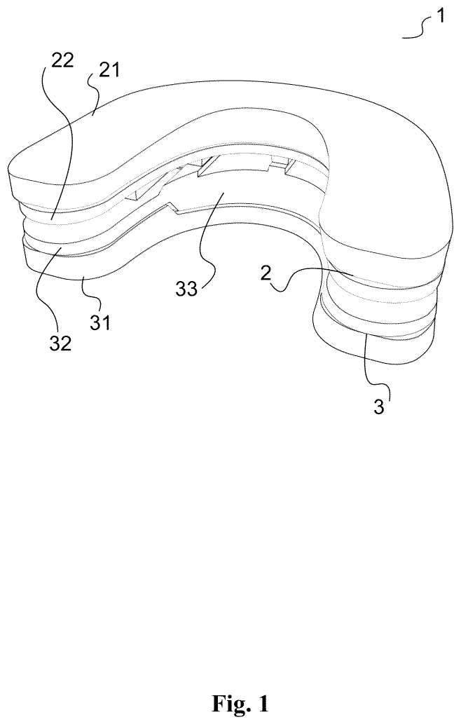

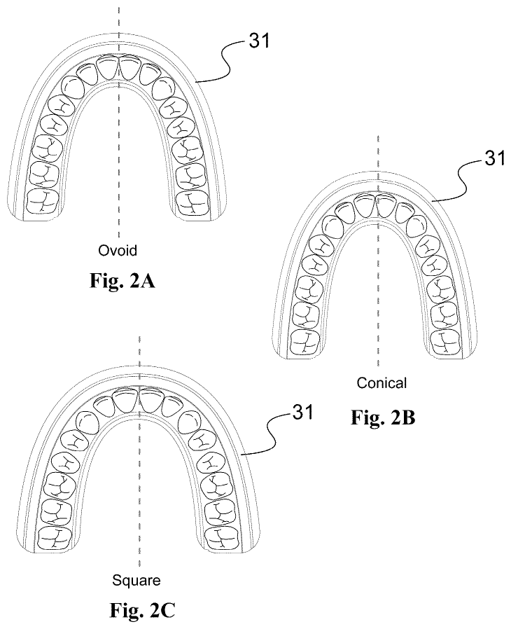

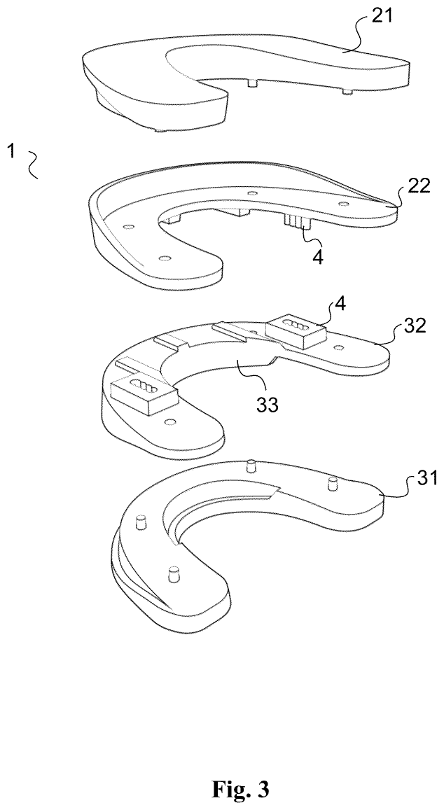

Specifically, referring to FIGS. 1 to 6, the mandibular advancement device 1 of the present disclosure includes an upper tray assembly 2 and a lower tray assembly 3. The upper tray assembly 2 has a generally arcuate outer shape to conform to the upper dental arch curve of the patient and is configured to be positioned adjacent to the upper dental arch when located in the patient's oral cavity. The lower tray assembly 3 also has a generally arcuate outer shape to conform to the lower dental arch curve of the patient and is configured to be positioned adjacent to the lower dental arch when located in the patient's oral cavity. Additionally, when the mandibular advancement device 1 is positioned in the patient's oral cavity, it is divided into a side closer to the lip side and a side closer to the tongue side in the front-to-back direction. The upper moldable component 21 and the lower moldable component 31 are configured to at least partially conform to the patient's upper and lower teeth during use, respectively. The shape of the patient's dental arch can generally be categorized into several shapes, including ovoid arch, conical arch, and square arch. Therefore, the arcuate shapes of the upper and lower moldable components 21, 31 include at least one of these shapes (as shown in FIGS. 2A, 2B, and 2C). Additionally, the upper tray assembly 2 and the lower tray assembly 3 can be composed of two completely separate parts or can be connected by a hinge or any other means, forming an inseparable unit.

Furthermore, the upper tray assembly 2 includes an upper moldable component 21 and an upper frame 22, while the lower tray assembly 3 includes a lower moldable component 31 and a lower frame 32. The upper moldable component 21 and the lower moldable component 31 can be detachably connected to the upper frame 22 and the lower frame 32, respectively, or can be fixed and non-detachable. The detachable connection forms can include protrusions and slot-type snap-fittings and/or additional components such as bolt latches, screws, pins, magnets, etc. For better fixation and convenience of the mandibular advancement device 1, it is preferred that the upper moldable component 21 and the lower moldable component 31 are connected to the upper frame 22 and the lower frame 32, respectively, using snap-fitting connections. These snap-fitting connections typically involve protrusions on the upper moldable component 21 and the lower moldable component 31 that match with slots on the upper lower frame 22 and the lower frame 32, respectively, or protrusions on the upper lower frame 22 and the lower frame 32 that match with slots on the upper moldable component 21 and the lower moldable component 31. The protrusions and slots can be symmetrically distributed near the middle part (i.e., the position of the mandibular advancement device 1 close to the patient's incisors) and/or on both ends (i.e., the position of the mandibular advancement device 1 near the patient's temporomandibular joint). Additionally, to prevent the separation of the upper tray assembly 2 and the lower tray assembly 3 during use and to ensure effective treatment, the connection of the upper tray assembly 2 and the lower tray assembly 3 during patient's use is primarily formed by the connection of the upper frame 22 and the lower frame 32, which are configured to connect and support the upper moldable component 21 and the lower moldable component 31, respectively. Typically, the upper frame 22 and the lower frame 32 are fixed together using snap-fitting connections. The specific form of the snap-fitting connections consists of at least one protrusion on either the upper frame 22 or the lower frame 32 and a corresponding groove on the other frame to accommodate the protrusion. Other forms of fixation can also be used in addition to snap-fittings, including but not limited to: threaded connections, hook connections, piston connections, welded connections, plug connections, pin connections, adhesive connections, spring connections, mortise and tenon connections, or elastic band connections. The connection method can involve a single form or a combination of two or more forms. The appropriate connection method depends on specific design requirements, material properties, and usage environment, ensuring a secure, reliable, and durable connection that is easy to install during production and convenient for patients to adjust.

The main usage process of the mandibular advancement device 1 involves the following steps: the upper moldable component 21 is connected to the upper frame 22 to form the upper tray assembly 2, and the lower moldable component 31 is connected to the lower frame 32 to form the lower tray assembly 3. By adjusting the relative position between the upper tray assembly 2 and the lower tray assembly 3, the patient's mandible is pulled away from the tongue side to open the patient's airway. This relative position adjustment between the upper tray assembly 2 and the lower tray assembly 3 is achieved by adjusting the upper frame 22 and the lower frame 32 through a position adjustment module 4 located between them. The position adjustment module 4 has two or more adjustment positions, allowing patients to select the position that best suit their comfort. The position adjustment module 4 can take various forms, generally located near the tongue side of the upper frame 22 and the lower frame 32, although it can be positioned elsewhere in special cases. The position adjustment module 4 might consist of a protrusion on the upper frame 22 that fits into a groove on the lower frame 32, or a protrusion on the lower frame 32 that fits into a groove on the upper frame 22. In other situations, the position adjustment module 4 may be a snap-fitting form that does not involve protrusions and grooves. It is evident that the position adjustment module 4 can also have other forms other than the snap-fitting form. There can be multiple fixed connections between the upper frame 22 and the lower frame 32 in addition to the position adjustment module 4 to provide extra stability to the upper frame 22 and the lower frame 32; or upper frame 22 and the lower frame 32 may be secured solely by the position adjustment module 4.

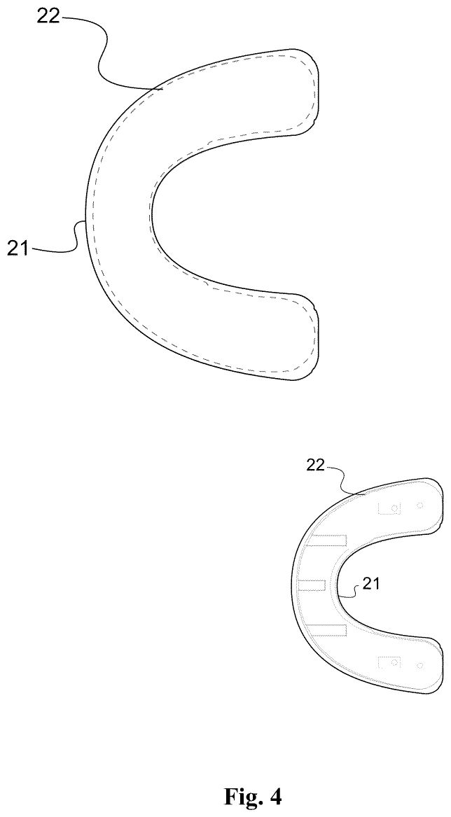

In the disclosure discussed herein, the dimensions of the upper and lower moldable components 21, 31 and the upper and lower frames 22, 32 are specified within a certain range. The projected area of the upper moldable component 21 and the lower moldable component 31 on the horizontal plane is greater than the projected area of the upper frame 22 and the lower frame 32 on the horizontal plane (as shown in FIG. 4), providing greater comfort and adjustability. Firstly, the upper moldable component 21 and the lower moldable component 31 use more flexible materials compared to the upper frame 22 and the lower frame 32, which helps to reduce irritation and abrasion to the oral soft tissues. Secondly, considering the variation in the distance from the inner side of the lips to the posterior molar area among different patients, an excessively long device might cause abrasions to the oral tissues behind the molars, especially in the joint area, potentially affecting occlusal function. For individuals where the dimensions of the upper moldable component 21 and the lower moldable component 31 are not suitable, the components can be trimmed and adjusted in length according to personal needs and preferences, enhancing comfort and personalized adaptability. This design not only ensures the functionality of the mandibular advancement device 1 but also grants patients more autonomy for adjustment. Similarly, based on this principle, the maximum height of the support baffle 33 is configured to be less than or equal to the maximum height of the lower moldable component 31, to prevent irritation and abrasion to the oral soft tissues.

The materials for the upper tray assembly 2 and the lower tray assembly 3 have a broad selection range. Since the upper moldable component 21 and the lower moldable component 31 are components primarily contact the patient's teeth and the gums during use, and the upper frame 22 and the lower frame 32 provide support, fixation, and positioning within the mandibular advancement device 1, it is essential that the upper tray assembly 2 and lower tray assembly 3 are made from at least two different materials. This ensures that the hardness of the material used for the upper frame 22 and the lower frame 32 is at least partially greater than the hardness of the material used for the upper moldable component 21 and the lower moldable component 31. The hardness of the materials for the upper frame 22 and the lower frame 32 ranges from Shore D45 to Shore D95. In some implementations, the upper tray assembly 2 and the lower tray assembly 3 may be made from a single material that is processed in a specific manner to achieve varying material rigidities. When selecting materials for the mandibular advancement device 1, factors such as biocompatibility, durability, functional stability, patient comfort, and ease of cleaning are extensively considered to select plastics or polymer materials that are suitable for oral medical devices and can be molded. Suitable materials include, but are not limited to: polypropylene (PP), polycarbonate (PC), polyethylene terephthalate (PET), polymethyl methacrylate (PMMA), polyphenylsulfone (PPSU), polyethylene (PE), high-density polyethylene (HDPE), low-density polyethylene (LDPE), ethylene-vinyl acetate (EVA), thermoplastic polyurethane (TPU), styrene-ethylene-butylene-styrene (SEBS), styrene-ethylene-propylene-styrene (SEPS), and silicone rubber.

The upper moldable component 21 and the lower moldable component 31 include a moldable material with a deformation temperature of less than 100 degrees Celsius. The upper frame 22 and lower frame 32 include a high-temperature stable material with a deformation temperature above 100 degrees Celsius. Specifically, the upper moldable component 21 and the lower moldable component 31 comprise a flexible thermoplastic material. These components are configured to soften when heated, allowing them to be adjusted and molded to match the patient's upper and lower dental arches when the patient bites down. During use, the patient simply needs to place the upper tray assembly 2 and the lower tray assembly 3 in hot water to heat them. Then, the patient bites down on the upper tray assembly 2 and the lower tray assembly 3, allowing the assemblies to form a good fit with the teeth. This biting process molds the heated upper moldable component 21 and the lower moldable component 31 to conform to the dental arches. The flexible thermoplastic material has a certain degree of flexibility and can withstand repeated mechanical stress without failing, which is crucial for the patient's use as they need to bite down on the moldable components of the mandibular advancement device 1. Additionally, the flexible thermoplastic material is lightweight and environmentally friendly.

The upper frame 22 and the lower frame 32 need to be made of a more stable material compared to the upper moldable component 21 and the lower moldable component 31. The material must have high-temperature stability to maintain their original shape after molding, providing structural support for the mandibular advancement device 1. Typically, the upper frame 22 and the lower frame 32 need to retain their shape over prolonged use, so they are made from a more durable material than the upper moldable component 21 and the lower moldable component 31. If the upper frame 22 and the lower frame 32 are also made from thermoplastic materials, their deformation temperature will be higher than that of the upper moldable component 21 and the lower moldable component 31. This ensures that the upper frame 22 and the lower frame 32 remain unaffected during the molding process of the upper moldable component 21 and the lower moldable component 31.

The following embodiments illustrate several structures of the mandibular advancement device 1 of the present disclosure in detail.

### Embodiment 1

In this embodiment, the mandibular advancement device 1 includes an upper tray assembly 2, which has a generally arcuate outer shape to conform to the patient's upper dental arch curve, and a lower tray assembly 3, which also has a generally arcuate outer shape to conform to the the patient's lower dental arch curve. The upper tray assembly 2 includes an upper moldable component 21 and an upper frame 22; the lower tray assembly 3 includes a lower moldable component 31 and a lower frame 32.

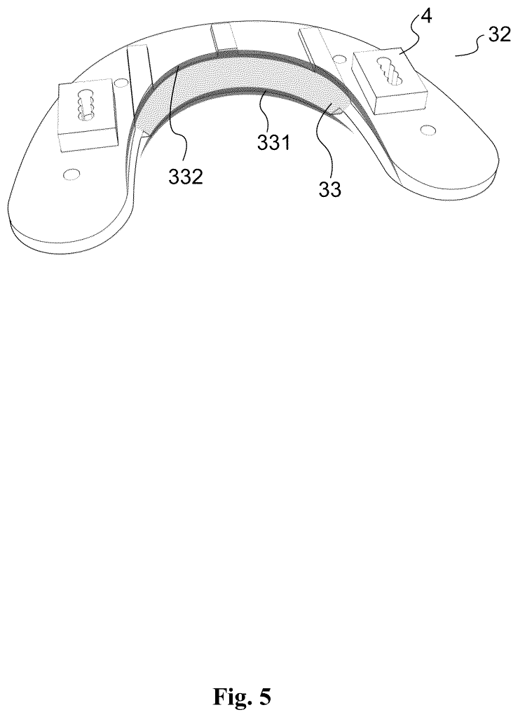

The lower frame 32 includes a support baffle 33, which is positioned near the tongue side of the lower frame 32 during use. The support baffle 33 has a top edge 331 and a bottom edge 332 (as shown in FIG. 5).

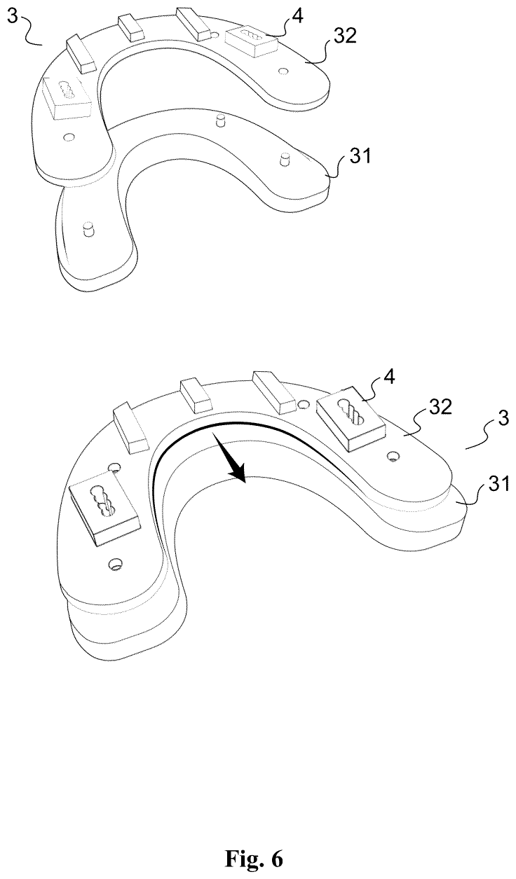

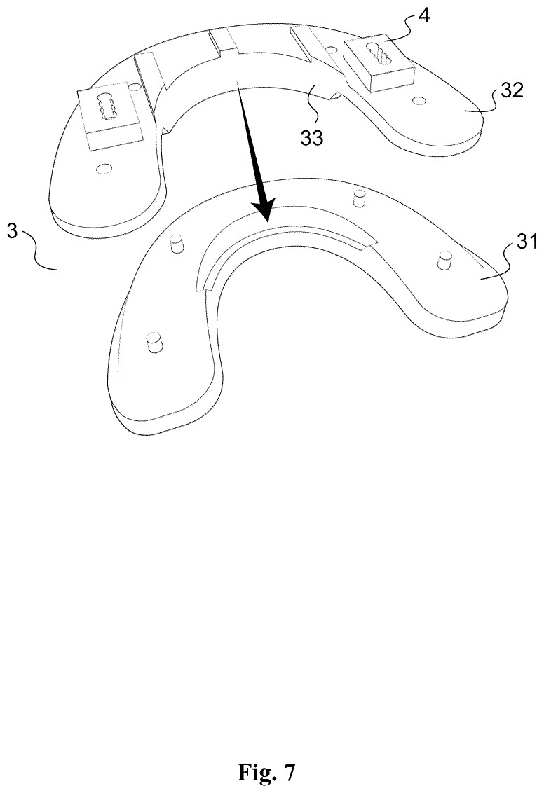

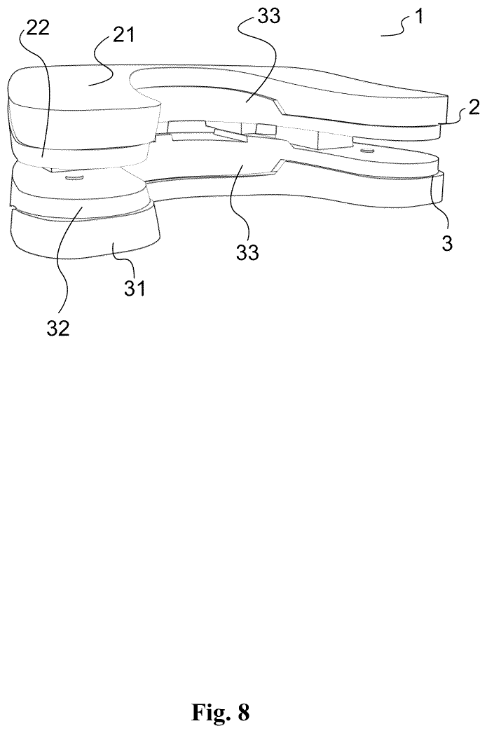

The primary function of the mandibular advancement device 1 is to pull the patient's mandible forward (away from the tongue side). However, due to habit, the patient's mandible tends to exert a backward force (towards the tongue side) on the lower moldable component 31. Since the lower moldable component 31 is made of flexible material, it is prone to significant deformation or misalignment or separation from the lower frame 32 (as shown in FIG. 6). The support baffle 33 helps to counteract the deforming force on the lower moldable component 31, maintaining its original shape, and secures the lower moldable component 31 and lower frame 32 of the mandibular advancement device 1, preventing them from detaching due to mechanical stress or habitual use. A stable and secure connection between the lower frame 32 and the lower moldable component 31 ensures the continuous and effective forward traction of the mandible, preventing airway obstruction caused by the failure of the mandibular advancement device 1 during the patient's sleep. This directly impacts the therapeutic effectiveness of the mandibular advancement device 1. A mandibular advancement device 1 with a support baffle 33 can more effectively alleviate the symptoms of obstructive sleep apnea syndrome. In some cases, the lower moldable component 31 has a shape that engages and matches the support baffle 33 (as shown in FIG. 7) to optimize the volume and comfort of the mandibular advancement device 1. Specifically, the close fit between the lower moldable component 31 and the support baffle 33 ensures that the volume of the lower tray assembly 3 does not increase due to the addition of the support baffle 33. This close-fitting design not only ensures the stability of the components during wear but also effectively reduces the sensation of a foreign object in the mouth. The support baffle 33 can be independently provided on either the upper frame 22 or the lower frame 32, with the preferred option being on the lower frame 32. Evidently, the support baffle 33 can also be simultaneously provided on both the upper frame 22 and the lower frame 32 (as shown in FIG. 8).

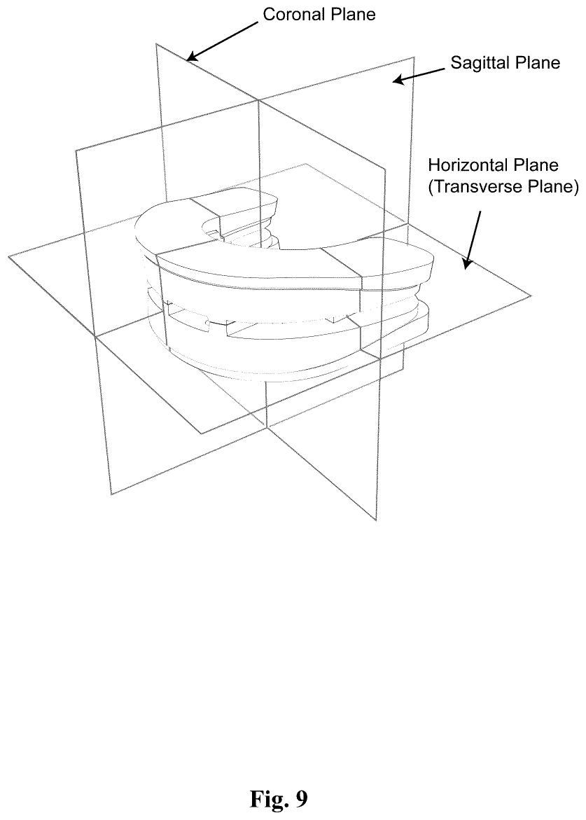

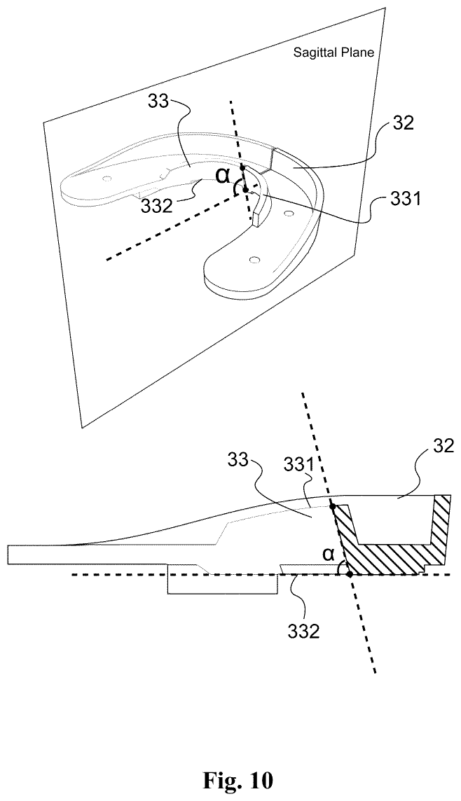

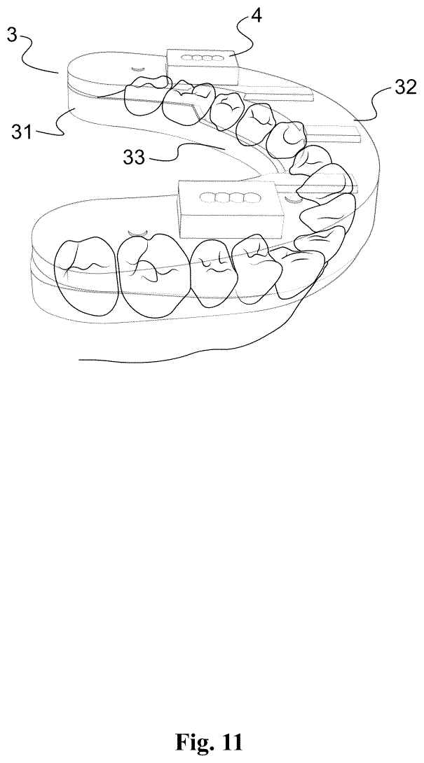

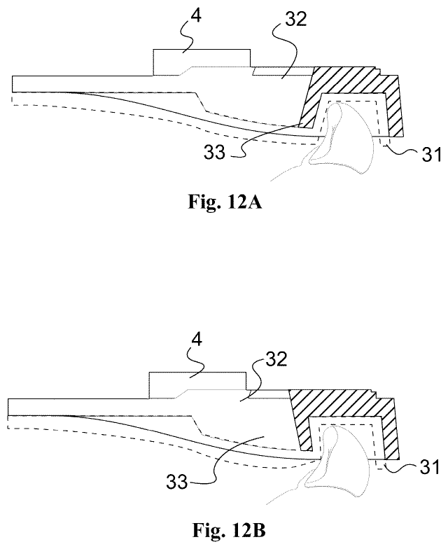

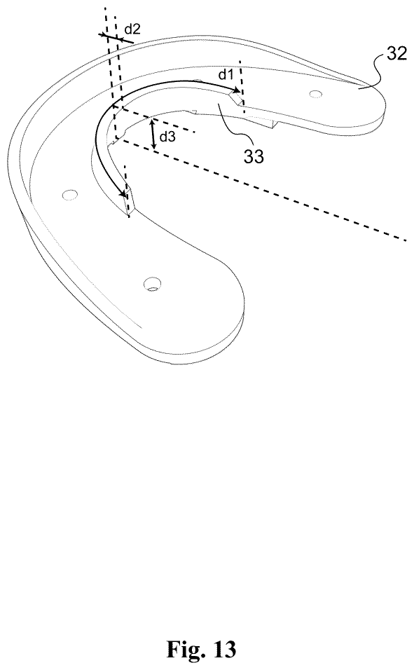

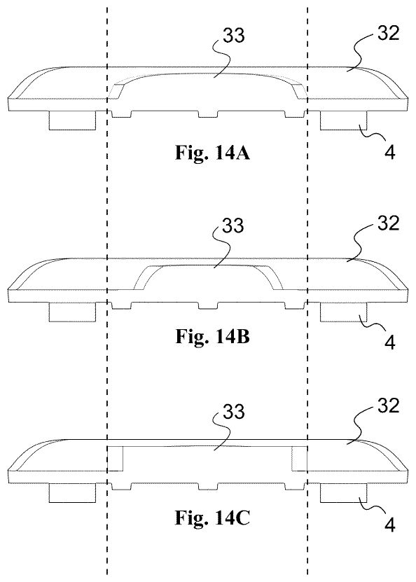

In this embodiment, the support baffle 33 is configured to have an incline. The support baffle 33 conforms to the shape of the mandible to provide better fit and support, further enhancing the patient's comfort during use. In this embodiment, the angle between the line connecting the intersections of the top edge 331 and the bottom edge 332 with a same sagittal plane of the lower frame 32 and the horizontal plane is between 20° to 150°, with a preferred range of 45° to 90° for greater comfort (as shown in FIGS. 9 and 10). This angle setting aims to improve patient comfort. Since the lower tray assembly 3 engages with the patient's mandible when using the mandibular advancement device 1 (as shown in FIG. 11), an angle inclined towards the tongue side helps to better match the lower tray assembly 3 with the patient's mandible (as shown in FIG. 12A). Conversely, an angle inclined away from the tongue side can cause the device to press against the patient's teeth and gums (as shown in FIG. 12B), potentially creating discomfort due to the stress formed at the sharp corners of the lower moldable component 31 during the therapeutic treatment. To ensure the support baffle 33 functions effectively during use, the length of the bottom edge 332 of the support baffle 33 should be greater than 10% of the overall curve length at the bottom edge 332 of the lower frame 32. Placing the support baffle 33 in the middle part of the lower frame 32 is optimal because the overall shape of the lower frame 32 is arcuate, and excessively long edges would result in ineffective areas. Considering the lengths of lower frames in existing mandibular advancement devices on the market, the preferred length of the support baffle 33 in this embodiment is greater than or equal to 5 mm (as shown by d1 in FIG. 13). Due to material and size constraints, the thickness of the support baffle 33 is set between 0.3 mm and 10 mm (as shown by d2 in FIG. 13) to provide adequate support for the moldable part within a suitable range. Typically, the support baffle 33 does not extend beyond the end of the patient's gums, so the height of the support baffle 33 is set to be less than or equal to 15 mm (as shown by d3 in FIG. 13). In addition to the aforementioned specifications, the hardness of the support baffle 33 needs to be defined. This embodiment does not restrict the specific material of the support baffle 33, as long as the material is harmless to the patient's health. The support baffle 33 can be made of any material. However, to ensure that the support baffle 33 can fulfill its function of supporting the lower moldable component 31 during use, its hardness must be specified. An overly hard support baffle 33 may scratch the patient's oral cavity, while an overly soft support baffle 33 may not provide adequate support. In this embodiment, based on the results of different hardness ranges for the support baffle 33 obtained through multiple repeated tests and professional instrument measurements, the hardness range of the support baffle 33 is set between Shore D45 and Shore D95, with Shore D65 to Shore D85 being the most suitable choice for effectiveness and comfort. Additionally, since the shape of the lower teeth and gums gradually decreases from the middle to the sides, the support baffle 33 is also configured to be higher in the middle and lower on both sides (as shown in FIG. 14A) to better fit the patient's oral cavity. In one implementation, the top edge 331 and bottom edge 332 of the support baffle 33 have continuous curvature. Evidently, the top edge 331 of the support baffle 33 can also have non-continuous curvature. Furthermore, the length of the support baffle 33 can be adjusted according to actual needs, and it can be relatively long or short (as shown in FIG. 14B).

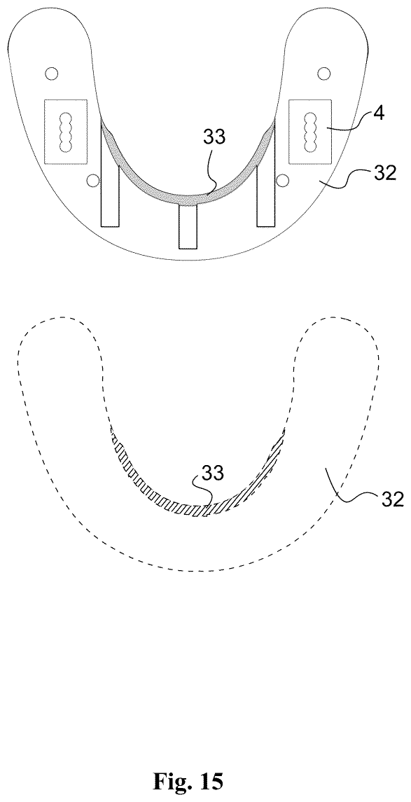

In other embodiments, in an orthogonal view from below, the support baffle 33 has a shaded area, and the ratio of this shaded area to the area of the lower frame 32 in the orthogonal view from below is less than or equal to 2:5 (as shown in FIG. 15). Additionally, the angle between the line connecting the intersections of the top edge 331 and the bottom edge 332 of the support baffle 33 with a same sagittal plane of the lower frame 32 and the horizontal plane is less than 90°, meaning the support baffle 33 can only incline towards the tongue side. This design ensures the effectiveness of the support baffle 33 while matching the slope angle of the lower teeth and gums, ensuring the patient's comfort during wear.

In other embodiments, the support baffle 33 can have a form where the top edge 331 is on the same horizontal plane and extends vertically towards the bottom edge 332 at certain positions, forming two side edges. Additionally, the support baffle 33 can have varying lengths (as shown in FIG. 14C).

### Embodiment 2

This embodiment of the mandibular advancement device 1 includes an upper tray assembly 2, which has a generally arcuate outer shape to conform to the upper dental arch curve of the patient, and a lower tray assembly 3, which also has a generally arcuate outer shape to conform to the lower dental arch curve of the patient. The upper tray assembly 2 includes an upper moldable component 21 and an upper frame 22; the lower tray assembly 3 includes a lower moldable component 31 and a lower frame 32.

The lower frame 32 includes a support baffle 33, which is configured to be positioned near the tongue side of the lower frame 32 during use.

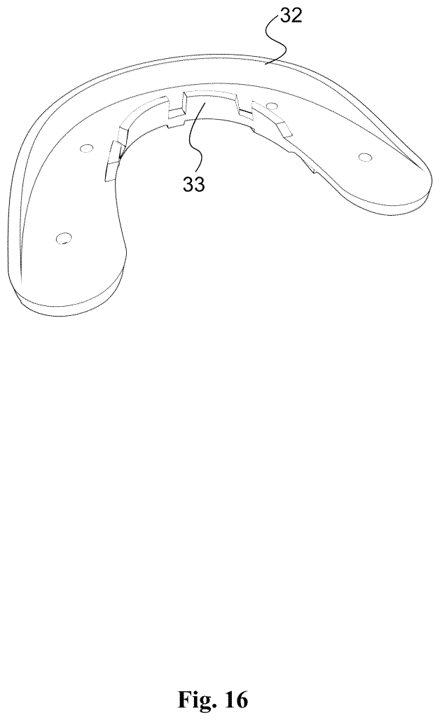

The difference between this embodiment and Embodiment 1 is that the support baffle 33 is divided into multiple spaced-apart sections. The support baffle 33 is not continuous but has an intermittent wall structure, while maintaining its function (as shown in FIG. 16). The intermittent wall structure reduces the material usage of the support baffle 33 to some extent, making the entire device lighter and more comfortable to wear. Additionally, the intermittent wall structure increases the flexibility of the mandibular advancement device 1, allowing it to better adapt to the patient's mandibular shape and movement. This reduces the pressure sensation of the support baffle 33 on the oral cavity and teeth during wear. If the angle of the support baffle 33 at the time of manufacture is not the most comfortable angle for the patient, the intermittent support baffle 33 can be more flexibly adjusted according to the patient's oral structure, achieving a personalized fit. This adjustability ensures that each patient can find the angle and size that best suit them, further enhancing comfort and therapeutic efficacy. Furthermore, the intermittent wall structure also brings additional advantages, such as improved breathability of the device, which can reduce the feeling of stuffiness during wear.

Moreover, the technical features in the above embodiments can be combined as needed to obtain a mandibular advancement device 1 that includes all or part of the aforementioned technical features. It must be noted that as used herein and in the appended claims, the regular forms “a” “an” “the” include their plural equivalents, unless the context clearly dictates otherwise.

The mandibular advancement device of the present disclosure has at least the following beneficial effects:

(1) Using a mandibular advancement device to prevent or reduce snoring and/or obstructive sleep apnea during sleep is a good conservative therapy alternative to CPAP. The current market offers various mandibular advancement devices with different designs, classified into moldable and non-moldable mandibular advancement devices based on their adjustability and adaptability during use. This disclosure pertains to a moldable mandibular advancement device. Non-moldable mandibular advancement devices require customization under the guidance of professional doctors. Since they are customized, they can perfectly match the patient's teeth and oral conditions, providing better comfort for the patient. However, due to the need for professional guidance and adjustment for the customized devices, the treatment cost of non-moldable mandibular advancement devices is relatively high, making them expensive. For some patients, the process of coordinating with doctors and customization is relatively complex and troublesome. Compared to non-moldable mandibular advancement devices, these patients, as well as those with limited budgets, might prefer purchasing moldable mandibular advancement devices. Moldable mandibular advancement devices allow patients to achieve a customized fit through a simple heating and biting process. They are relatively cheaper than customized devices, making them a more economical choice for most patients.

(2) Although moldable mandibular advancement devices are more likely to be chosen by most patients compared to non-moldable ones, they still have significant problems and limitations. Currently, patient reviews for moldable mandibular advancement devices on the market are generally poor, with low satisfaction levels mainly due to the need for improvements in comfort and reliability. For example, the primary function of the lower tray assembly is to pull the patient's mandible away from the tongue side. However, because this goes against the force of inertia, the patient's mandible exerts a force on the lower tray assembly in the opposite direction of the mandibular advancement device's traction force. Existing mandibular advancement devices without support baffles lack structures that provide support to the upper and lower moldable components to counteract the force exerted by the mandible on the lower moldable component. Since the upper and lower moldable components include flexible materials, this force easily causes deformation, particularly in the lower moldable component. Deformation of the lower moldable component leads to the backward sliding of the mandible, rendering the device ineffective. Additionally, some current mandibular advancement devices tend to dislodge at the connection points between the upper and lower frames and the upper and lower moldable components, especially in the lower tray assembly. The lower moldable component and the lower frame are often connected by small snap-fitting structures, resulting in an insecure attachment. Due to the force exerted by the mandible on the lower moldable component and the flexibility of its moldable material, these snap-fittings can easily detach, causing separation between the lower moldable component and the lower frame. This disclosure, through comparison with existing designs, innovatively introduces a mandibular advancement device with a support baffle, which prevents the separation of the lower moldable component from the lower frame. Compared to existing mandibular advancement devices on the market, this innovative design offers greater convenience for patients during use and facilitates better patient compliance. This key innovation addresses critical issues in the current technology, providing an advantageous and novel solution.

(3) For patients, the mandibular advancement device of this disclosure not only prevents the displacement of the lower moldable component from the lower frame but also includes an angled support baffle. Typically, the gums and lower teeth are not vertical but have a sloped profile. The angled support baffle matches the slope of the gums and teeth, allowing the device to fit more closely to the patient's oral cavity and providing greater comfort during use. Comfort is a crucial factor for patients when purchasing a device. If the device is uncomfortable or affects treatment by causing issues such as component detachment, patients may be reluctant to wear it for extended periods, thus impacting treatment efficacy. Therefore, this disclosure improves patient compliance to some extent, ensuring more stable treatment. Moreover, the disclosure specifies various parameters to enhance the effectiveness of the support baffle during use. For instance, by defining the angle and height of the support baffle, the device can be made suitable for a wide range of patients. The angle of the support baffle is set within a range of 20° to 150°, preferably 45° to 90°, and the height is set to be less than or equal to 15 mm. If the support baffle extends outward at too great an angle or is too tall, it may exceed its basic position and restrict the occlusion of the upper and lower jaws. Conversely, if the support baffle is angled inward too much or is too short, it may lose its effectiveness. This disclosure also specifies the length of the support baffle as needed. Since the actual growth pattern of teeth and gums is higher in the middle and lower on both sides, and due to the autonomous movement of the patient's mandible towards the tongue side, the force exerted on the lower tray assembly mainly concentrates in the middle part. Therefore, placing the support baffle in the middle section of the lower frame is the optimal arrangement. If the baffle extends too far on both sides, it essentially becomes an ineffective area. Additionally, setting the hardness of the support baffle is a crucial aspect. As long as the support baffle is safe for the patient, no specific material requirements are imposed, allowing the baffle to be made of any material that complies with safety regulations. However, to ensure the support baffle functions effectively as part of the mandibular advancement device, it must be able to support the lower moldable component. Given this requirement, the hardness of the support baffle is specified to be between Shore D45 to Shore D95. Within this range, overly hard support baffles can injure the patient's oral cavity, while overly soft baffles cannot provide adequate support. Therefore, the preferred hardness range is Shore D65 to Shore D85, making it a more suitable choice for use. All data in this disclosure are selected through multiple measurements with professional machines, providing scientifically based support baffle, ensuring both effectiveness and comfort for the patient. This effective design of the support baffle distinguishes it from existing mandibular advancement devices, offering superior performance and a better experience for patients.

(4) For the product itself, mandibular advancement devices without a support baffle often cause the lower moldable component to detach, and because the upper and lower moldable components include flexible materials, they are highly susceptible to damage. Damage to the product not only reduces the lifespan of the mandibular advancement device but also leads to instability, thereby diminishing its therapeutic effectiveness. The mandibular advancement device with a support baffle of this disclosure, significantly enhances the stability of the device by reinforcing the fixation between the lower moldable component and the lower frame. This design effectively prevents opposing forces from pulling on the lower moldable component, ensuring it does not easily detach or get damaged during use. This improvement not only makes the mandibular advancement device more durable but also ensures the stability and reliability of its therapeutic effect.

1. A mandibular advancement device with a support baffle, the mandibular advancement device comprising:
an upper tray assembly, configured to have an arcuate outer shape to conform to an upper dental arch curve of a patient;
a lower tray assembly, configured to have an arcuate outer shape to conform to a lower dental arch curve of the patient;
wherein the upper tray assembly includes an upper moldable component and an upper frame, wherein the upper moldable component has a projected area greater than a projected area of the upper frame;
wherein the lower tray assembly includes a lower moldable component and a lower frame, wherein the lower moldable component has a projected area greater than a projected area of the lower frame;
wherein the lower frame includes the support baffle, configured to be positioned on the lower frame near a tongue side during use;
wherein the support baffle has a top edge and a bottom edge, and a line connecting intersections of the top edge and the bottom edge with a same sagittal plane of the lower frame forms an angle with a horizontal plane between 20° to 150°,
wherein a hardness of the support baffle is between Shore D45 to Shore D85,
wherein a thickness of the support baffle is between 0.3 mm to 10 mm, and
wherein the support baffle is configured to counteract a deforming force on the lower moldable component and support the lower moldable component.

2. The mandibular advancement device according to claim 1, wherein arcuate shapes of the upper moldable component and the lower moldable component include at least one of an ovoid arch shape, a conical arch shape, and a square arch shape.

3. The mandibular advancement device according to claim 1, wherein the upper moldable component and the lower moldable component are configured to at least partially conform to the upper dental arch and the lower dental arch of the patient during use, respectively.

4. The mandibular advancement device according to claim 1, wherein a length of the bottom edge of the support baffle is greater than 10% of an overall curve length at a bottom edge of the lower frame.

5. The mandibular advancement device according to claim 1, wherein the upper moldable component and the lower moldable component include a flexible thermoplastic material.

6. The mandibular advancement device according to claim 1, wherein a hardness of a material of the upper frame and the lower frame is at least partially greater than a hardness of a material of the upper moldable component and the lower moldable component.

7. A mandibular advancement device with a support baffle, the mandibular advancement device comprising:
an upper tray assembly, configured to be positioned adjacent to an upper dental arch of a patient when located in the patient's oral cavity;
a lower tray assembly, configured to be positioned adjacent to a lower dental arch of the patient when located in the patient's oral cavity;
wherein the upper tray assembly includes an upper moldable component and an upper frame, wherein the upper moldable component has a projected area greater than a projected area of the upper frame;
wherein the lower tray assembly includes a lower moldable component and a lower frame, wherein the lower moldable component has a projected area greater than a projected area of the lower frame;
wherein the lower frame includes the support baffle, configured to be positioned on the lower frame near a tongue side during use; and
wherein, in an orthogonal view from below, the support baffle has a shaded area, and a ratio of the shaded area to an area of the lower frame in the orthogonal view from below is less than or equal to 2:5, and wherein a top edge of the support baffle slants from the lower frame,
wherein a hardness of the support baffle is between Shore D45 to Shore D85,
wherein a thickness of the support baffle is between 0.3 mm to 10 mm, and
wherein the support baffle is configured to counteract a deforming force on the lower moldable component and support the lower moldable component.

8. The mandibular advancement device according to claim 7, wherein the support baffle is divided into multiple spaced-apart sections.

9. The mandibular advancement device according to claim 7, wherein the lower moldable component has a shape that engages and matches the support baffle.

10. The mandibular advancement device according to claim 7, wherein the upper moldable component and the lower moldable component are configured to soften when heated, to allow them to be adjusted and molded to respectively match the upper dental arch and the lower dental arch of the patient upon biting down.

11. The mandibular advancement device according to claim 7, wherein the upper moldable component and the lower moldable component include a moldable material having a deformation temperature of less than 100 degrees Celsius.

12. The mandibular advancement device according to claim 11, wherein the upper frame and the lower frame include a high-temperature stable material having a deformation temperature greater than 100 degrees Celsius.

13. A mandibular advancement device with a support baffle, the mandibular advancement device comprising:
an upper tray assembly, configured to be positioned adjacent to an upper dental arch of a patient when located in the patient's oral cavity;
a lower tray assembly, configured to be positioned adjacent to a lower dental arch of the patient when located in the patient's oral cavity;
wherein the upper tray assembly includes an upper moldable component and an upper frame, wherein the upper moldable component has a projected area greater than a projected area of the upper frame;
wherein the lower tray assembly includes a lower moldable component and a lower frame, wherein the lower moldable component has a projected area greater than a projected area of the lower frame;
wherein the lower frame includes the support baffle, configured to be positioned on the lower frame near a tongue side during use;
wherein, in an orthogonal view from below, the support baffle has a shaded area, and a ratio of the shaded area to an area of the lower frame in the orthogonal view from below is less than or equal to 2:5;
wherein the support baffle has a shape that is higher in a middle and lower on both sides, and a top edge of the support baffle slants from the lower frame;
wherein a thickness of the support baffle is between 0.3 mm to 10 mm; and
wherein the support baffle is configured to counteract a deforming force on the lower moldable component and support the lower moldable component.

14. The mandibular advancement device according to claim 13, wherein the upper frame and the lower frame are configured to engage and support the upper moldable component and the lower moldable component, respectively.

15. The mandibular advancement device according to claim 13, wherein a position adjustment module is provided between the upper frame and the lower frame.

16. The mandibular advancement device according to claim 15, wherein the position adjustment module has two or more adjustment positions.

17. The mandibular advancement device according to claim 13, wherein a maximum height of the support baffle is less than or equal to a maximum height of the lower moldable component.

18. A mandibular advancement device with a support baffle, the mandibular advancement device comprising:
an upper tray assembly, configured to be positioned adjacent to an upper dental arch of a patient when located in the patient's oral cavity;
a lower tray assembly, configured to be positioned adjacent to a lower dental arch of the patient when located in the patient's oral cavity;
wherein the upper tray assembly includes an upper moldable component and an upper frame, wherein the upper moldable component has a projected area greater than a projected area of the upper frame;
wherein the lower tray assembly includes a lower moldable component and a lower frame, wherein the lower moldable component has a projected area greater than a projected area of the lower frame;
wherein the lower frame includes the support baffle, configured to be positioned on the lower frame near a tongue side during use;
wherein a thickness of the support baffle is between 0.3 mm to 10 mm;
wherein the support baffle is configured to counteract a deforming force on the lower moldable component and support the lower moldable component; and
wherein the support baffle has one or more of the following characteristics:
A. having a top edge and a bottom edge, and a line connecting intersections of the top edge and the bottom edge with a same sagittal plane of the lower frame forms an angle with a horizontal plane between 20° to 150°;
B. a length of the top edge of the support baffle being greater than or equal to 5 mm;
C. a vertical height of the support baffle being less than or equal to 15 mm.

19. The mandibular advancement device according to claim 18, wherein the top edge and the bottom edge of the support baffle have continuous curvature.

20. The mandibular advancement device according to claim 18, wherein the upper tray assembly and the lower tray assembly include at least two different materials.

21. (canceled)

22. The mandibular advancement device according to claim 18, wherein the lower moldable component has a shape that engages and matches the support baffle.
